(19) United States
(12) Patent Application Publication
(10) Pub. No.: US 2006/0276766 A1
(43) Pub. Date: Dec. 7, 2006

(54) Pad for use with thong garment

(76) Inventor: Glenda Kentolall, Katy, TX (US)

Correspondence Address: ALTON W. PAYNE, 5508 GRAND LAKE, HOUSTON, TX, 77081, US

(21) Appl. No.: 11/146,840

(22) Filed: Jun. 7, 2005

(51) Int. Cl. A61F 13/15

(52) U.S. Cl. 604/385.01; 604/385.16

(57) ABSTRACT

The present invention relates generally to disposable absorbent articles, sanitary napkins, pads and the like. Specifically, the present invention relates to a disposable absorbent article, sanitary napkin or pad for use with thong garments.

### FIELD OF THE INVENTION

The present invention relates generally to disposable absorbent articles, sanitary napkins, pads and the like. Specifically, the present invention relates to a disposable absorbent article, sanitary napkin or pad for use with thong garments.

### BACKGROUND OF THE INVENTION

Disposable absorbent sanitary articles, e.g., such as pantiliner pads, frequently make use of one or more lines or grooves to deter the egress of liquid out of the article. For example, U.S. Pat. No. 5,807,365 (Luceri) discloses a disposable shield of a generally dog-bone shape comprising a thin, highly absorbent pad having a body-contacting surface, an absorbent layer, a liquid barrier, a positioning adhesive for attaching the pad to an undergarment, and a release layer to protect the adhesive prior to use. The pad further comprises densified areas forming unbroken concentric rings having the same general shape as the shield itself. These rings are made by fusing all pad layers together in a pattern embosser to create densified areas resistant to the flow of liquid. The densified areas are made contiguous such that fluid, when introduced or deposited on the pad, will be prevented or hindered from flowing to the edges of the pad. Moreover, the concentric rings serve to separate, or compartmentalize, the pad into distinct absorbing areas which are isolated from each other.

U.S. Pat. No. 5,795,344 (Chappefi) discloses an absorbent article, such as a sanitary napkin having a cover, a baffle and an absorbent between the cover and baffle and includes a single, unbroken embossed channel positioned inward from the peripheral edge of the article. The channel impedes the flow of fluid toward the edges of the absorbent article and increases absorbent utilization in the absorbent article. The channel can be produced various ways, such as by application of heat, including hot calendar embossing or by using ultrasonic means.

U.S. Pat. No. 5,891,118 (Toyoshima et al) discloses elongated absorbent articles that includes an anti-leakage groove formed along each longitudinal side portion of the article. The anti-leakage groove may be continuous or discontinuous and only extends partially into the thickness of the article.

Other United States Letters Patents disclosing absorbent articles or pads with channels, grooves or embossed or de-bossed lines are: U.S. Pat. No. 4,624,666 (DeRossett et al), U.S. Pat. No. 4,758,240 (Glassman), U.S. Pat. No. 5,104,396 (Oatleyetal), U.S. Pat. No. 5,447,506 (Lindquist), U.S. Pat. No. 5,451,442 (Pieniak et al) and U.S. Pat. No. 5,613,960 (Mizutani). Various United States Design patents also disclose absorbent articles or pads with channels, grooves or embossed or de-bossed lines, such as U.S. Design Pat. No. 272,189 (Sneider) and U.S. Design Pat. No. 272,190 (Sneider). Pantiliners or other sanitary disposable absorbent articles or pads are also commercially available that make use of at least one barrier line to prevent the egress of liquid from a peripheral portion of the article.

While all of the foregoing absorbent articles are suitable for their intended purposes, they never the less leave something to be desired from the standpoint of retention of fluid, (e.g., resistance to leakage) flexibility and conformability to the body of the wearer.

The prior art does include various Pantiliners for use with thong shaped garments. For example, U.S. Design Pat. No. 366,524 (Chung) discloses a thong pantiliner that has one bulbous end that tapers down to a narrower, slightly arced end. The width of the bulbous end is approximately two and one half times that of the narrow end. The bottom view has a pattern of four undulating lines on its surface.

U.S. Design Pat. No. 424,195 (Talon) discloses a thong pantiliner that is generally heart shaped with a top having two lobes separated by a distinct point and two substantially straight sides angling down to an end. The end has a rounded point having a radius less than half of the radius of the lobes. The top view has a “quilting” pattern of diamonds with flowers at the corners of the diamonds.

U.S. Design Pat. No. 392,736 (Erickson) discloses a pantiliner somewhat similar in overall shape (in the top view) to that of U.S. Design Pat. No. 366,524 (Chuog). However, the comers are somewhat sharper and the bottom is not arced, but is straight. The top view has a pattern of undulating lines (different than that of the Chung patent). The side views (FIGS. 4 and 5) indicate a two-step surface where a bottom layer is longer than a top layer.

U.S. Design Pat. No. 431,293 (Finkle et al.) discloses a feminine hygiene sanitary pad that is thong shaped with an overall shape (in the top view) similar to that of U.S. Des. Pat. No. 366,524 (Chung) and U.S. Des. Pat. No. 392,736 (Erickson). Here, however, the top surface slopes downwardly adjacent the edges as can be seen in FIGS. 1 and 2-5.

U.S. Pat. No. 5,683,373 (Darby) discloses a sanitary napkin shaped for use with a thong garment. The pad is v-shaped in plan view, bulbous at first top end having a predetermined width, and tapering therefrom longitudinally to a midportion, and from there longitudinally narrowing in an elongated lower end portion terminating in a second end. The lower end portion has a substantially uniform width, less than twenty-five percent of the width of the bulbous first end portion and a length greater than one third, but less than one half the total length of the pad from longitudinal end to end.

U.S. Pat. No. 5,729,835 (Williams) is directed to a pantiliner for use with thong shaped underwear having an upper first layer fabricated of a cotton panel, a second layer fabricated of a cotton weave, a third layer fabricated of an absorbent material, a fourth layer fabricated of a cotton weave, a fifth layer fabricated of a plastic material, a sixth layer fabricated of a cotton panel, an adhesive strip, and a protective backing for the adhesive strip.

Other thong shaped pantiliners are disclosed in the following United States design patents: U.S. Des. Pat. No. 276,184 (Whitehead), U.S. Des. Pat. No. 368,519 (Harrison et al), U.S. Des. Pat. No. 394,503 (Perrini), U.S. Des. Pat No. 411,006 (Nixon et al), U.S. Des. Pat. No. 416,324 (Nixon et al), U.S. Des. Pat. No. 425,196 (Nixon et al), and U.S. Pat. No. 3,805,790 (Kaczmarzyk et al).

While the aforementioned thong shaped pantiliners are generally suitable for their intended purposes they never the less leave something to be desired from the standpoint of leakage.

It is, therefore, a feature of the present invention to provide a disposable absorbent article, sanitary napkin or pad for use with thong garments.

A feature of the present invention is to provide a pad for use with thong garments having a shape with the same thickness from the bulb-shaped top to the gradual narrowing of the narrowed portion.

Another feature of the present invention is to provide a pad for use with thong garments that can be worn without a tampon for a regular heavy cycle.

Another feature of the present invention is to provide a pad for use with thong garments that requires the disposal of one item instead of two, e.g., tampon & panty liner.

Another feature of the present invention is to provide a pad for use with thong garments whereby a longer pad can be made for night use.

Yet another feature of the invention is to provide a pad for use with thong garments which has a thick pad for a heavy cycle.

Still another feature of the present invention is utilizing a pad for use with thong garments which has a medium thickness pad for beginning of cycle or end of cycle.

Another feature of the present invention is to provide a pad for use with thong garments that is quick and easy to change when active and on the go.

Yet another feature of the present invention is to provide a pad for use with thong garments that require fewer items to pack.

Still another feature of the present invention is to provide a pad for use with thong garments that fits in a small package.

Still another feature of the present invention is to provide a pad for use with thong garments that is less conspicuous with tight clothing.

Yet still another feature of the present invention is to provide a pad for use with thong garments less likely to spread disease and germs because there is no contact with flesh when inserting or removing the pad.

Yet further, an additional feature of the present invention is to provide a pad for use with thong garments that requires only one size panty.

Yet further, another feature of the present invention is to provide a pad for use with thong garments that feels and looks good with maximum protection fewer feminine products to carry, store and dispose of.

Still, further another feature of the present invention is to provide a pad for use with thong garments that has adhesive on back side, and will stay secure with any thong panty.

Yet another feature of the present invention is to provide to provide a pad for use with thong garments especially when tampons can be irritating to the vagina, causing the use of pads and thus limiting the style of panty to be worn during the cycle, however with a thicker thong pad, thong panties can be worn through an entire cycle.

Yet still another feature of the present invention is to provide a pad for use with thong garments such that the thong pad fits closer to body and provides more secure protection than the conventional sanitary napkin.

Additional features and advantages of the invention will be set forth in part in the description which follows, and in part will become apparent from the description, or may be learned by practice of the invention. The features and advantages of the invention may be realized by means of the combinations and steps particularly pointed out in the appended claims.

### SUMMARY OF THE INVENTION

To achieve the foregoing objects, features, and advantages and in accordance with the purpose of the invention as embodied and broadly described herein, a pad for use with thong garments is provided.

### BRIEF DESCRIPTION OF THE DRAWINGS

The accompanying drawings which are incorporated in and constitute a part of the specification, illustrate a preferred embodiment of the invention and together with the general description of the invention given above and the detailed description of the preferred embodiment given below, serve to explain the principles of the invention.

FIG. 1 is a plan view of a preferred embodiment of the pad for use with a thong garment encompassed by the present invention.

FIG. 2 is an elevation view of a preferred embodiment of the pad for use with a thong garment encompassed by the present invention as illustrated in of FIG. 1.

FIG. 3 is an elevation view of another preferred embodiment of the pad for use with a thong garment encompassed by the present invention as illustrated in of FIG. 1.

FIG. 4 is an elevation view of yet another preferred embodiment of the pad for use with a thong garment encompassed by the present invention as illustrated in of FIG. 1.

FIG. 5 is a plan view of a preferred embodiment of the pad having variable lengths for use with a thong garment encompassed by the present invention.

FIG. 6 is an elevation view of a preferred embodiment of the pad for use with a thong garment encompassed by the present invention as illustrated in of FIG. 5.

FIG. 7 is an elevation view of another preferred embodiment of the pad for use with a thong garment encompassed by the present invention as illustrated in of FIG. 5.

FIG. 8 is an elevation view of yet another preferred embodiment of the pad for use with a thong garment encompassed by the present invention as illustrated in of FIG. 5.

FIG. 9 is a plan view of a preferred embodiment of the pointed pad for use with a thong garment encompassed by the present invention.

FIG. 10 is an elevation view of a preferred embodiment of the pad for use with a thong garment encompassed by the present invention as illustrated in of FIG. 9.

FIG. 11 is an elevation view of another preferred embodiment of the pad for use with a thong garment encompassed by the present invention as illustrated in of FIG. 9.

FIG. 12 is an elevation view of yet another preferred embodiment of the pad for use with a thong garment encompassed by the present invention as illustrated in of FIG. 9.

FIG. 13 is a perspective view of a preferred embodiment of the pad for use with a thong garment encompassed by the present invention.

FIG. 14 is a perspective, cut-away view of a preferred embodiment of the pad for use with a thong garment encompassed by the present invention.

The above general description and the following detailed description are merely illustrative of the generic invention, and additional modes, advantages, and particulars of this invention will be readily suggested to those skilled in the art without departing from the spirit and scope of the invention.

### DETAILED DESCRIPTION OF THE PREFERRED EMBODIMENTS

Reference will now be made in detail to the present preferred embodiments of the invention as described in the accompanying drawings.

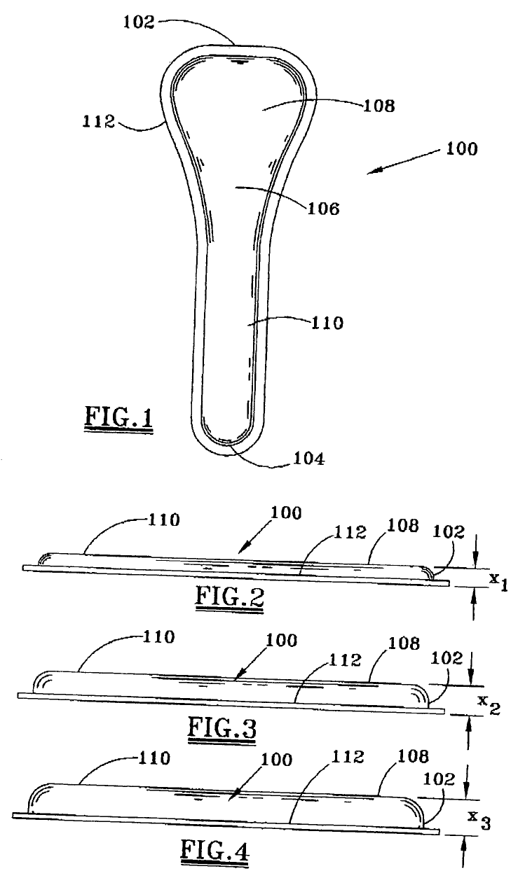

FIG. 1 is a plan view of a preferred embodiment of the pad 100 for use with a thong garment encompassed by the present invention. The pad 100 has an expanded end 102, a narrowed end 104 and a transition section 106 there between. Around the perimeter of the pad 100 is a planar edge 112. The expanded end 102 has an expanded surface 108 and the narrowed end 104 has a narrowed surface 1 10.

FIG. 2 is an elevation view of a preferred embodiment of the pad 100 for use with a thong garment encompassed by the present invention as illustrated in of FIG. 1. The narrowed surface 1 10 is illustrated remote from the expanded surface 108. The planar edge 112 is around the entire perimeter of the pad 100. The pad 100 has a thickness of x₁.

FIG. 3 is an elevation view of another preferred embodiment of the pad 100 for use with a thong garment encompassed by the present invention as illustrated in of FIG. 1. The narrowed surface 110 is illustrated remote from the expanded surface 108. The planar edge 112 is around the entire perimeter of the pad 100. The pad 100 has a thickness of x₂. The thickness of x₂ is thicker than the thickness x₁.

FIG. 4 is an elevation view of yet another preferred embodiment of the pad for use with a thong garment encompassed by the present invention as illustrated in of FIG. 1. The narrowed surface 110 is illustrated remote from the expanded surface 108. The planar edge 112 is around the entire perimeter of the pad 100. The pad 100 has a thickness of x₃. The thickness of x₃ is thicker than the thickness x₂.

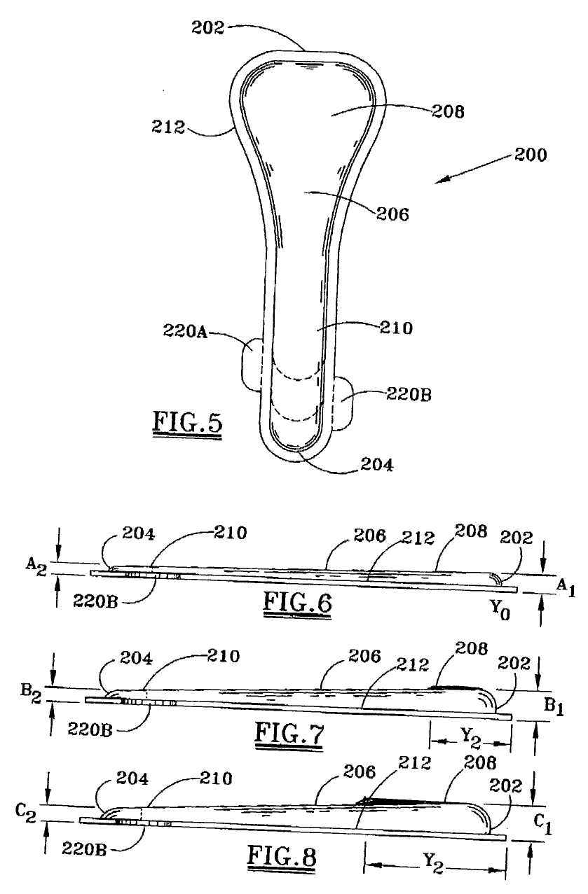

FIG. 5 is a plan view of a preferred embodiment of the pad 200 having variable lengths for use with a thong garment encompassed by the present invention. The pad 200 has an expanded end 202, a narrowed end 204 and a transition section 206 there between. Around the perimeter of the pad 200 is a planar edge 212. The expanded end 202 has an expanded surface 208 and the narrowed end 204 has a narrowed surface 210. The narrowed end 204 has one or more perforations (see FIG. 5, dashed lines). The perforations (dashed lines) provide that the narrowed end 204 can be severed at one or more locations so as to create a pad 200 of varying length. Further, the pad 200 has one or more tabs 220A, 220B. The tabs 220A, 220B are provided to wrap around the thong garment to better secure the pad 200 to the garment.

FIG. 6 is an elevation view of a preferred embodiment of the pad for use with a thong garment encompassed by the present invention as illustrated in of FIG. 5. The narrowed surface 210 is illustrated remote from the expanded surface 208. The tabs 220A, 220B are associated with the narrowed surface 210. The planar edge 212 is around the perimeter of the pad 200. The pad 200 has a thickness of A₁. The embodiment of the pad 200 illustrated in FIG. 6 has a gradual decreasing thickness beginning at y₀ where the thickness is A₁. The decreasing thickness of the pad can be a constant decreasing rate of thickness. Alternately, the decreasing thickness of the pad 200 can be a varying rate of thickness. The thickness of the pad at the narrowed end 204 is A₂ which is less than A₁, the thickness of the pad at the expanded end 202.

FIG. 7 is an elevation view of another preferred embodiment of the pad for use with a thong garment encompassed by the present invention. The narrowed end 204 and the narrowed surface 210 are illustrated remote from the expanded end 202 and the expanded surface 208. The planar edge 212 is around the perimeter of the pad. At the expanded end 202, the pad has a thickness of B₁. The thickness of B₁ is thicker than the thickness A₁. The embodiment of the pad illustrated in FIG. 7 has a constant thickness for the distance Y₁ remote from the expanded end 202, where the thickness is B₁. Thus, the expanded surface 208 has a constant thickness B₁ for the distance Y₁. And after the distance Y₁ remote from the expanded end 202, the pad has a gradual decreasing thickness. The decreasing thickness of the pad can be a constant decreasing rate of thickness. Alternately, the decreasing thickness of the pad can be at a varying rate of thickness. The thickness of the pad at the narrowed end 204 is B₂ which is less than B₁, the thickness of the pad at the expanded end 202.

FIG. 8 is an elevation view of yet another preferred embodiment of the pad for use with a thong garment encompassed by the present invention. The narrowed end 204 and the narrowed surface 210 are illustrated remote from the expanded end 202 and the expanded surface 208. The planar edge 212 is around the perimeter of the pad. At the expanded end 202, the pad has a thickness of C₁. The thickness of C₁ is thicker than the thickness B₁. The embodiment of the pad illustrated in FIG. 8 has a constant thickness for the distance Y₂ remote from the expanded end 202, where the thickness is C₁. Thus, the expanded surface 208 has a constant thickness C₁ for the distance Y₂. And after the distance Y₂ remote from the expanded end 202, the pad has a gradual decreasing thickness. The decreasing thickness of the pad can be a constant decreasing rate of thickness. Alternately, the decreasing thickness of the pad can be a varying rate of thickness. The thickness of the pad at the narrowed end 204 is C₂ which is less than C₁, the thickness of the pad at the expanded end 202.

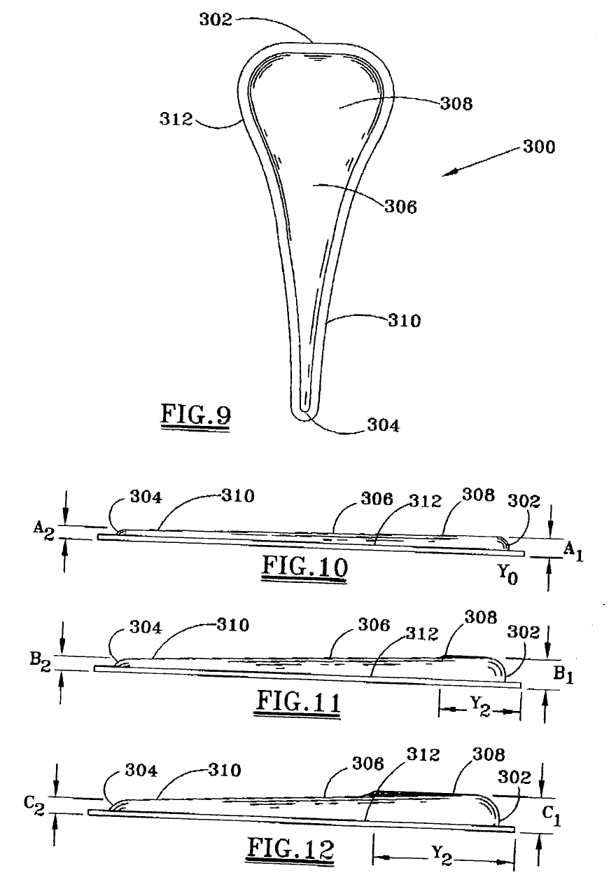

FIG. 9 is a plan view of a preferred embodiment of the pad 300 for use with a thong garment encompassed by the present invention. The pad 300 has an expanded end 302, a narrowed end 304 and a transition section 306 there between. Around the perimeter of the pad 300 is a planar edge 312. The expanded end 302 has an expanded surface 308 and the narrowed end 304 has a narrowed surface 310. The narrowed end 304 is tapered to a point 304. Optionally although not illustrated, the pad 300 can have one or more tabs 220A, 220B as illustrated in FIGS. 5-8. The tabs 220A, 220B are provided to wrap around the thong garment to better secure the pad 200 to the garment.

FIG. 10 is an elevation view of a preferred embodiment of the pad for use with a thong garment encompassed by the present invention as illustrated in of FIG. 5. The narrowed surface 310 is illustrated remote from the expanded surface 308. The planar edge 312 is around the perimeter of the pad. The pad has a thickness of A₁. The embodiment of the pad illustrated in FIG. 10 has a gradual decreasing thickness beginning at y₀ where the thickness is A₁. The decreasing thickness of the pad can be a constant decreasing rate of thickness, or, the decreasing thickness of the pad can be a varying rate of thickness. The thickness of the pad at the narrowed end 304 is A₂ which is less than A₁, the thickness of the pad at the expanded end 302.

FIG. 11 is an elevation view of another preferred embodiment of the pad for use with a thong garment encompassed by the present invention. The narrowed end 304 and the narrowed surface 310 are illustrated remote from the expanded end 302 and the expanded surface 308. The planar edge 312 is around the perimeter of the pad. At the expanded end 302, the pad has a thickness of B₁. The thickness of B₁ is thicker than the thickness A₁. The embodiment of the pad illustrated in FIG. 7 has a constant thickness for the distance Y₁ remote from the expanded end 302, where the thickness is B₁. Thus, the expanded surface 308 has a constant thickness B₁ for the distance Y₁. And after the distance Y₁ remote from the expanded end 302, the pad has a decreasing thickness. The decreasing thickness of the pad can be a constant decreasing rate of thickness, or, alternately, the decreasing thickness of the pad can be at a varying rate of thickness. The thickness of the pad at the narrowed end 304 is B₂ which is less than B₁, the thickness of the pad at the expanded end 302.

FIG. 12 is an elevation view of yet another preferred embodiment of the pad for use with a thong garment encompassed by the present invention. The narrowed end 304 and the narrowed surface 310 are illustrated remote from the expanded end 302 and the expanded surface 308. The planar edge 312 is around the perimeter of the pad. At the expanded end 302, the pad has a thickness of C₁. The thickness of C₁ is thicker than the thickness B₁. The embodiment of the pad illustrated in FIG. 8 has a constant thickness for the distance Y₂ remote from the expanded end 202, where the thickness is C₁. Thus, the expanded surface 308 has a constant thickness C₁ for the distance Y₂. And after the distance Y₂ remote from the expanded end 302, the pad has a gradual decreasing thickness. The decreasing thickness of the pad can be a constant decreasing rate of thickness, or, alternately, the decreasing thickness of the pad can be a varying rate of thickness. The thickness of the pad at the narrowed end 304 is C₂ which is less than C₁, the thickness of the pad at the expanded end 202.

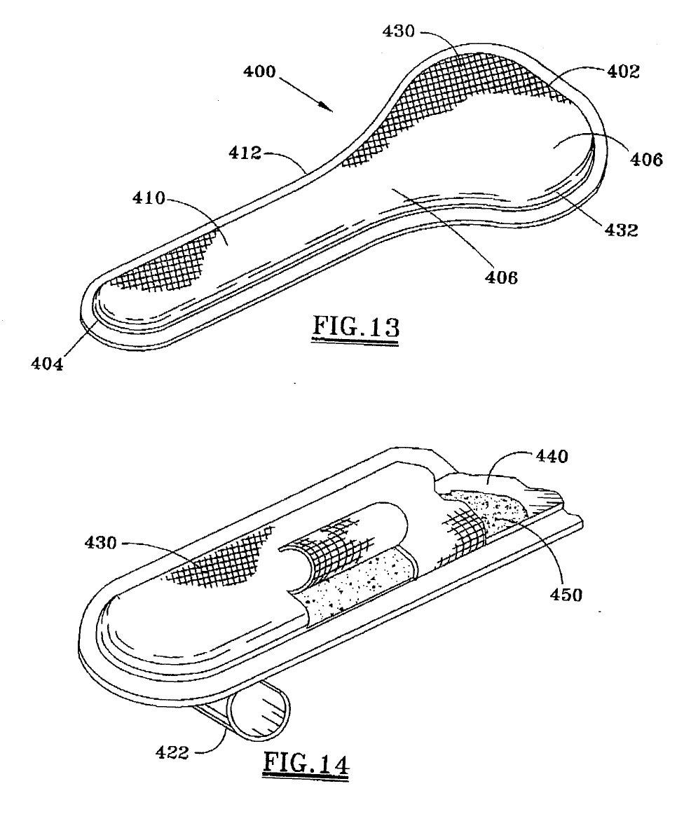

FIG. 13 is a perspective view of a preferred embodiment of the pad 400 for use with a thong garment encompassed by the present invention. The pad 400 has an expanded end 402, a narrowed end 404 and a transition section 406 there between. Around the perimeter of the pad 400 is a planar edge 412. The expanded end 402 has an expanded surface 408 and the narrowed end 104 has a narrowed surface 110.

FIG. 14 is a perspective, cut-away view of a preferred embodiment of the pad 400 for use with a thong garment encompassed by the present invention as illustrated in FIG. 13. An impervious outer layer 440 provides that fluids are not transferred to the thong garment. Adjacent to the impervious outer layer 440 is an absorbent inner layer 450 for absorbing and containing fluids. A pervious outer layer 430 provides a conduit for the transfer of fluid through the pervious outer layer 430 and to the absorbent inner layer 450. Essentially all the fluid is contained between the pervious outer layer 430 and the impervious outer layer 440 to be contained in the absorbent inner layer 450.

Additional advantages and modification will readily occur to those skilled in the art. The invention in its broader aspects is therefore not limited to the specific details, representative apparatus, and the illustrative examples shown and described herein. Accordingly, the departures may be made from the details without departing from the spirit or scope of the disclosed general inventive concept.

1. A pad for use with a thong garment worn by a person comprising
(a) a pervious outer layer for engaging the person,
(b) an impervious outer layer for engaging the thong garment,
(c) an absorbing inner layer contained between the pervious outer layer and the impervious outer layer,
the combination of the pervious outer layer, the impervious outer layer and absorbing inner layer comprising:
(1) an expanded planar end having a constant thickness between the pervious outer layer and the impervious outer layer,
(2) a narrowed planar end remote from the expanded end having a gradual deliberate decreasing thickness between the pervious outer layer and the impervious outer layer that is less than the thickness associated with the expanded end, and
(3) a transition section between the expanded end and the narrowed end where the thickness has a deliberate and gradual decrease.

2. A pad for use with a thong garment worn by a person comprising
(a) a pervious outer layer for engaging the person,
(b) an impervious outer layer for engaging the thong garment,
(c) an absorbing inner layer contained between the pervious outer layer and the impervious outer layer,
the combination of the pervious outer layer, the impervious outer layer and absorbing inner layer comprising:
(1) an expanded planar end having a constant thickness between the pervious outer layer and the impervious outer layer having a portion of the thickness being constant, and
(2) a narrowed planar end remote from the expanded planar end having a gradual deliberate decreasing thickness between the pervious outer layer and the impervious outer layer that is less than the thickness associated with the expanded end such that the respective narrowing and the thickness are directly proportional and the narrowing section ends in an apex remote from the expanded section.

3. The pad for use with a thong garment worn by a person as defined in claim 2 wherein the thickness decreases at a deliberate, gradual, constant rate.

4. The pad for use with a thong garment worn by a person as defined in claim 2 wherein the thickness decreases at a deliberate, gradual, varying rate.
